(19) United States
(12) Patent Application Publication
(10) Pub. No.: US 2021/0290799 A1
(43) Pub. Date: Sep. 23, 2021

(54) CORONA DISCHARGE BASED TREATMENTS OF ARTICLES

(71) Applicant: University of South Florida, Tampa, FL (US)

(72) Inventors: Ying Zhong, Tampa, FL (US); Libin Ye, Tampa, FL (US); Siriam Sundar Shankara Narayanan, Tampa, FL (US); Vladislav Paley, Tampa, FL (US); Xudong Wang, Tampa, FL (US)

(21) Appl. No.: 17/210,184

(22) Filed: Mar. 23, 2021

Related U.S. Application Data
(60) Provisional application No. 62/993,623, filed on Mar. 23, 2020.
(60) Provisional application No. 63/015,445, filed on Apr. 24, 2020.

(51) Int. Cl. A61L 2/14; A61L 2/26; H01T 19/04

(52) U.S. Cl. CPC A61L 2/14; A61L 2/26; H01T 19/04; A61L 2202/26; A41D 13/11; A61L 2202/16; A61L 2202/11; A61L 2202/122; A61L 2202/15

(57) ABSTRACT

A method for treating an article, the method including generating a corona discharge from an electrode; providing an article within the corona discharge; and exposing the article to the corona discharge to disinfect and/or electrically charge the article.

### PRIORITY CLAIM

This application claims benefit from U.S. Provisional Patent Application No. 62/993,623, filed Mar. 23, 2020, which claims benefit from U.S. Provisional Patent Application No. 63/015,445, filed Apr. 24, 2020, all of which are hereby incorporated by reference.

### TECHNICAL FIELD

This document relates to corona discharge based disinfecting treatment systems and related methods, for example, methods of using corona discharge treatment for disinfecting articles.

### BACKGROUND

During a pandemic, personal protective equipment (“PPE”), such as disposable articles and reusable masks, can be in reduced supply, for example high-grade respirator filters (e.g. N95 and N100). Such respirators are generally single-use because they often lose their effectiveness during use over a given duration of time. The disinfection and reuse of respirators, rather than their disposal, however, would ease production pressures and issues stemming from a supply shortage. Such reuse of masks can, however, put users at high risk of exposure to a disease or an infection if the masks are not properly disinfect.

During use, respirator filters can become loaded with accumulation of contaminants. The contamination can be any pathogen, such as bacteria, fungus, or viruses. Additionally, the charges injected into the fibers of the filters can be neutralized through respiration humidity during use, storage conditions, or charged contaminants. The neutralization of these charges reduces filtration efficacy of the filter. Disinfecting methods, such as washing, or applying UV radiation, alcohol, or heat, can remove the static attraction effect provided by the “injected” charges in the non-woven fibers thus decreasing the filtration efficiency.

### SUMMARY

This document relates to corona discharge based treatment systems and related methods, for example, methods of using corona discharge treatment for disinfecting articles, such as respirator filters (e.g. N95 and N100).

Respirators capable of filtering very small particles are commonly made of fine strands of plastic (e.g., polypropylene) blown onto a screen to create a complex netting. The process of blowing these strands is performed at a high voltage which injects charges into the fibers as they are being blown. The injected charges provide an electrostatic interaction between the non-woven fibers and any filtered particulates increasing the filtration efficacy. Manufacturers then take these filters of fine plastic fibers and form them into disposable masks or filters for use in reusable masks. Pore size and static charges are the two key factors for respirators to function as intended. After being worn for a certain period of time (e.g., 10 hours), the effectiveness of masks deteriorates because of the loss of static charges and/or contamination accumulation.

Corona discharge based treatment systems and methods of using corona discharge treatment for disinfecting articles masks described herein can advantageously disinfect and/or charge the mask for reuse without affecting the original microstructure. As a radiative treatment technology, a corona discharge based treatment method does not cause thermal or reactive damage to the treated materials, and requires less time and energy consumption compared to other commonly used treatment techniques (e.g., UV or gamma methods). The corona discharge based treatment methods and system provided herein can provide the following advantages.

First, corona discharge treatment is a non-contact method that ionizes atmospheric chemicals and inducing chemical reactions between the generated reactive species, filtration materials, and biologics present on the filtration material surfaces. Solvents or other chemical agents are not needed to contact the filter in a chemical wash or dipping method which can generate potentially hazardous waste.

Second, corona discharge is more energy efficient when compared with other non-contact radiative treatment methods, such as UV- or gamma exposure. A high voltage corona discharge is generated within several seconds and can disinfect pathogens within minutes that would take hours under UV light.

Third, corona discharge treatment does not require the heating, pressurization, or exposure to high moisture of the material and therefore avoids any issues related to heat or pressure. Although heat treatment of materials, objects, and surfaces is another common method of treatment, using thermal energy, steam, and pressure to disinfect surfaces, e.g., autoclaving, can create high energy and steam exposure that can cause significant damage to the microstructure of respirator filters. Heat and pressure treatments can also remove or diminish initial charges that were injected into the filter material, thereby neutralizing the treatment efficacy of the respirator filter.

Finally, corona discharge treatment to disinfect and recharge single-use, disposable medical protection equipment, such as surgical or respirator masks, provides increased personal protections for equipment users during situations of shortage or low production. Treating and reusing disposable equipment increases cost savings and decreases waste generated in situations where protective equipment is heavily used.

The corona discharge device treatment provides a means of disinfection as well as charge injection to the respirator filter material. This restores at least a portion filtration efficacy and sterility of an individual filter, thereby allowing reuse of previously disposable respirator filters. This allows health care and first response workers, and civilians, to reuse respirator filters in a safe, convenient and affordable manner with affecting the material microstructure. This can be useful in relieving PPE shortages. In addition to making masks reusable, the major and broader application corona treatment devices provides treatment solution for various articles and surfaces, which but not limit to small and large objects in homes, hospitals, labs, shared public areas, GMP facilities.

The production of disposable articles (e.g., gowns, gloves, or masks) utilizes disinfection and charge injection treatments. The corona discharge treatment devices and methods disclosed herein provide disinfection and charge injection simultaneously which reduces treatment times and increases production capacities. Corona discharge treatment devices and methods also eliminate the use of solid or liquid disinfection materials in the bulk production of disposable articles increasing the production efficiency by eliminated drying times per article.

In a first aspect, the disclosure provides a method for treating an article, the method including generating a corona discharge from an electrode; providing an article within the corona discharge; and exposing the article to the corona discharge to disinfect and/or electrically charge the article.

In some embodiments, the generating can include applying a voltage to the electrode of less than 100 kV. The generating can include applying a voltage selected from the group consisting of about 6 kV to about 40 kV, about 6 kV to about 10 kV, about 10 kV to about 20 kV, or about 20 kV to about 40 kV. The exposing can occur in a cycle, each cycle including a treatment time and a storage time, wherein the article can be exposed to the corona discharge during the treatment time, and wherein the article may not be exposed to the corona discharge during the storage time. The treatment time can be from about 15 s to about 10 mins. The storage time can be from about 1 s to 60 min.

In some embodiments, the exposing can occur in 2 cycles to 10 cycles. The exposing reduces a detectable biologic marker by a logarithmic power value in a range from 0.5 to 8. The article can be a mask or a filter. The exposing reduces or eliminates the presence of a bacteria, a spore, a fungus, a virus, or combinations thereof. The exposing increases a surface charge density of the article by at least 0.001 μC/m². The surface charge density of the article can be increased for at least 1 days. The exposing can include generating an ion density of about 10⁴ to about 10⁸ ions/cm³. The providing can include arranging the article or portions of the article between about 1 cm to about 50 cm from the electrode.

In a second aspect, the disclosure provides a system for treating an article, the system including a power supply or a means for connecting to a power supply; and a discharge electrode electrically coupled to the power supply; wherein the system is configured to generate a corona discharge from the discharge electrode to disinfect and/or electrically charge the article.

In some embodiments, the discharge electrode can include a needle, a plurality of needles, a wire, a plurality of wires, or a flat plate. The discharge electrode can have a tip having a curvature radius of about 0.1 μm to about 1500 μm. The system can include a portable device, wherein the portable device can be a hand-held device including one or more nozzles, each nozzle including the discharge electrode, and wherein the hand-held device including a handle configured for single hand. The system can include an enclosure including a housing configured for receiving the discharge electrode and a sample support structure configured to receive the article, wherein the discharge electrode and the article on the sample support structure are arranged within the enclosure housing such that the article can be spaced apart from the discharge electrode by an electrode-sample distance. The discharge electrode can include a wire shaped in the form of a ring, and wherein the system can include an electrode support structure configured to receive and position the wire by the electrode-sample distance from the article.

Unless otherwise defined, all technical and scientific terms used herein have the same meaning as commonly understood by one of ordinary skill in the art to which the subject matter of this disclosure pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

The patent application file contains at least one drawing executed in color. Copies of this patent or patent application publication with color drawings(s) will be provided by the Office upon request and payment of the necessary fee.

FIG. 1A is a schematic diagrams of an exemplary corona treatment system.

FIGS. 1B and 1C are schematic diagrams of example discharge electrodes with curvature radii shown.

FIG. 1D is a schematic diagram of a treatment area created by a corona treatment system.

FIG. 1E is a chart depicting a simulated corona discharge area using a wire discharge electrode.

FIGS. 2A and 2B are side-view schematic diagrams of a portable corona treatment system.

FIG. 2C is a perspective-view schematic diagram of a portable corona treatment system.

FIG. 3 is a side-view schematic diagram of a hand-held corona treatment system.

FIG. 4A is a schematic diagram of an article being processed in a corona disinfection system.

FIG. 4B and FIG. 4C are schematic diagrams of a contaminated article before and after being processed in a corona discharge system.

FIG. 5A depicts an image of an E. coli culture grown on a plate from a swabbed article that had not been processed in a corona disinfection system.

FIG. 5B depicts an image of an E. coli culture grown on a plate from a swabbed article that had been processed in a corona disinfection system.

FIG. 6A is a schematic diagram of the experimental corona treatment system setup.

FIGS. 6B and 6C are regular and exploded images of melt-blown polypropylene sheets used for samples, and in an N95 respirator mask, respectively.

FIG. 6D is a schematic diagram of the corona treatment system experimental setup.

FIG. 7A is a line graph comparing the logarithmic biologic reduction to discharge voltage.

FIG. 7B is a line graph comparing the electric field to electrode distance with an inset line graph comparing the electric field evaluated at the sample surface to discharge voltage.

FIG. 7C is a line graph comparing the ion density to discharge voltage.

FIG. 7D is a line graph comparing the relative intensity to wavelength of a reactive species at five electrode potentials.

FIG. 8A is a line graph comparing the logarithmic biologic reduction to electrode distance.

FIG. 8B is a line graph comparing the relative intensity to wavelength of a reactive species at six electrode potentials.

FIG. 8C is a line graph comparing the electric field to electrode distance with an inset line graph comparing the electric field evaluated at the sample surface to discharge voltage.

FIG. 8D is a line graph comparing the ion density to electrode distance.

FIG. 9A is a line graph comparing the logarithmic biologic reduction to treatment time.

FIG. 9B is a bar chart comparing the logarithmic biologic reduction to treatment time FIG. 10A is a bar chart comparing the logarithmic biologic reduction to electrode shape.

FIG. 10B is a line graph comparing the relative intensity to wavelength of a reactive species using two electrode shapes.

FIG. 10C is a bar chart comparing the logarithmic biologic reduction to two electrode shapes and two treatment cycle counts.

FIG. 10D is a line graph comparing ozone concentration to decay time for three treatment cycle counts.

FIG. 11A is a line graph comparing the logarithmic biologic reduction to treatment time.

FIG. 11B is a bar chart comparing the logarithmic biologic reduction to three treatment cycle counts.

FIG. 11C is a bar chart comparing the logarithmic biologic reduction to two environmental conditions.

FIG. 11D is a bar chart comparing the logarithmic biologic reduction to three biological sample types.

FIG. 12A is a scanning electron microscope image of an untreated E. coli sample.

FIG. 12B is a scanning electron microscope image of a treated E. coli sample.

FIG. 12C is a negative image of gel electrophoresis results comparing the digested DNA of the E. coli sample of FIG. 12B across several treatment times.

FIG. 12D is an image of gel electrophoresis results comparing the digested proteins of the E. coli sample of FIG. 12B across several treatment times.

FIG. 13A is a line graph comparing surface charge density across time for four treatment cycle counts.

FIG. 13B is a bar chart comparing surface charge density across time for four treatment cycle counts.

FIG. 13C is a bar chart comparing surface charge density to seven treatment cycle times.

### DETAILED DESCRIPTION

This document relates to corona discharge based treatment systems and related methods, for example, methods of using corona discharge treatment for treating articles, such as respirator filters (e.g. N95 and N100). The treatment systems and methods provided herein can advantageously treat respirator filters while providing a portable, cost-effective solution.

The corona treatment systems described herein can be used to disinfect an article by reducing microbial populations present. Examples of microbial populations include viral, bacterial, and fungal organisms, such as E. coli, B. subtilis, S. aureus, S. cerevisiae, Candida, Yersinia pestis, and their spores if applicable or SARS-CoV-2. The corona treatment systems reduce microbial populations by exposing the article to a corona discharge generated by an electric field. The corona discharge, and reactive species within, disrupt or damage the microbial population through one or more mechanisms, such as DNA or RNA damage, protein misfolding, or cell wall disruption.

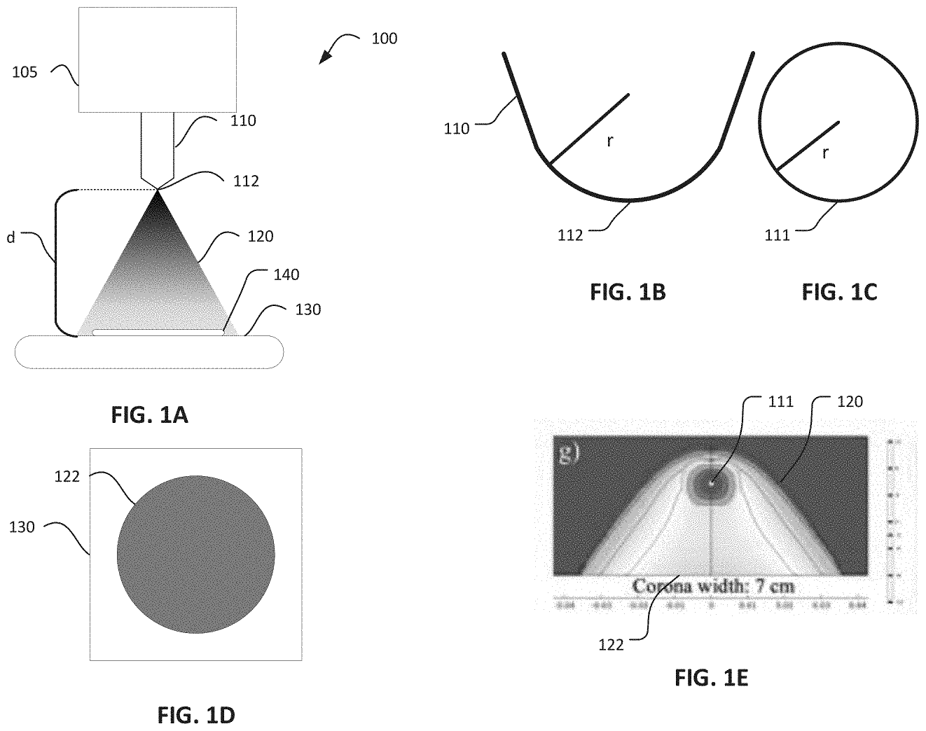

FIG. 1A shows an exemplary corona treatment system 100 for treating an article 140, such as a single-use respirator. The system 100 includes a corona discharge device 105 electrically coupled to a discharge electrode 110. The discharge device 105 includes, or is in electrical communication with, a power supply (e.g., a battery, an outlet, or a generator) which supplies electrical power to the discharge device 105 and can include AC, DC, or RF power. The discharge device 105 includes electrical components (e.g., transformers, rectifiers, and/or control circuitry) to sustain the discharge electrode 110 at a voltage (e.g., an electric potential) and operate the discharge electrode 110 as a positive electrode (e.g., an anode).

The corona treatment system 100 includes an opposing electrode 130 at a distance, d, from the discharge electrode 110, e.g., an electrode acting as the negative electrode (e.g., a cathode). The distance can depend on the power applied to an article within the corona discharge 120, the gas composition, discharge electrode 110 and/or opposing electrode 130 material or coatings, and can be in a range from about 1 cm to about 50 cm (e.g., about 1 cm to about 40 cm, about 1 cm to about 30 cm, about 10 cm to about 20 cm, about 10 cm to about 50 cm, about 20 cm to about 50 cm, about 30 cm to about 40 cm, or about 10 cm to about 40 cm). In some embodiments, the discharge device 105 sustains the opposing electrode 130 at a voltage and operates the opposing electrode 130 as the positive electrode and the discharge electrode 110 as the negative electrode.

The opposing electrode is generally composed of a non-reactive, conductive material such as stainless steel. In some embodiments, the opposing electrode 130 is composed of or coated in a dielectric, or insulating material such as a ceramic, glass, or polymer. In various example embodiments, the opposing electrode 130 is composed of a polymer such as polyactic acid (PLA), or PLA infused with a conductive material (e.g., conductive PLA). The opposing electrode 130 can be constructed using an additive manufacturing method, such as 3D printing.

In some embodiments, the voltage is in a range from about 5 k to about 100 kV (e.g., about 5 k to about 100 kV, about 20 k to about 100 kV, about 40 k to about 100 kV, about 60 k to about 100 kV, about 80 k to about 100 kV, about 5 k to about 70 kV, about 5 k to about 70 kV, or about 5 k to about 30 kV). In some embodiments, the voltage is in a range from about 6 kV to about 40 kV (e.g., about 6 kV to about 40 kV, about 10 kV to about 30 kV, about 20 kV to about 30 kV, or about 30 kV to about 40 kV). The discharge device 105 operates the discharge electrode 110 at a current of 20 mA or less (e.g., 15 mA or less, 10 mA or less, 5 mA or less, 4 mA or less, or 2 mA or less). In some embodiments, the discharge device 105 operates the discharge electrode 110 at a current of 1 mA or less (e.g., 1 mA or less, 0.8 mA or less, 0.6 mA or less, 0.4 mA or less, or 0.2 mA or less). In some embodiments, the discharge electrode 110 can be operated at a current of 0.05 mA or less (e.g., 0.05 mA or less, 0.03 mA or less, 0.02 mA or less, or 0.01 mA or less).

The discharge device 105 depicts a discharge electrode 110 in the shape of a needle with a conical, sharp tip 112. In general, the discharge electrode 110 is constructed of a conductive material, such as a metal or conductive composite. Non-limiting examples of discharge electrode 110 materials include steel, tungsten, titanium, cobalt, or combinations or alloys thereof. In various alternative embodiments, the conductive discharge electrode 110 is at least partially encased in a dielectric or insulating material, such as a ceramic or a polymer.

In general, the discharge electrode 110 includes a tip 112, point, or line with a curvature radius wherein applying the voltage to the discharge electrode 110 creates a high electric field and generates a corona discharge 120 between the tip 112 and the opposing electrode 130. FIG. 1B is an exploded schematic diagram of the tip 112 of discharge electrode 110. The tip 112 of the discharge electrode 110 includes a curvature radius, r, as a measurement of the tip 112 curvature. A larger r value determines a ‘flatter’ tip whereas a smaller r value is a sharper, finer tip 112. FIG. 1C is a schematic diagram of a cross-section of a wire-shaped discharge electrode 111.

In some embodiments, the discharge electrode 110 shape includes a wire, an edged-surface (e.g., a blade), a wedge point, or any combination thereof. In some embodiments, the discharge electrode 110 curvature radius is in a range from 0.1 μm to 1500 μm (e.g., 1 μm to 1500 μm, 10 μm to 1500 μm, 100 μm to 1500 μm, 1000 μm to 1500 μm, 1250 μm to 1500 μm, 0.1 μm to 1250 μm, 0.1 μm to 1000 μm, 0.1 μm to 100 μm, 0.1 μm to 10 μm, or 0.1 μm to 1 μm). In some embodiments, the discharge electrode 110 and opposing electrode 130 include a flat surfaces (e.g., planar) such as in dielectric barrier discharge.

Although the corona treatment system 100 of FIG. 1 depicts the use of a single discharge electrode 110, in some embodiments, more than one discharge electrodes 110 can be used (e.g., two, three, four, or more). In some embodiments, the more than one discharge electrodes 110 are arranged in an array, such as parallel needle-shaped discharge electrodes 110 arranged such that the tips 112 form a line or a plane.

The voltage difference between the discharge electrode 110 and opposing electrode 130 induces an electric field between the tip 112 of the discharge electrode 110 and the opposing electrode 130 which ionizes gas molecules in the interspatial area. The gas surrounding the discharge electrode 110 and opposing electrode 130 is atmospheric gas at ambient temperature. In some embodiments, the gas is an elemental or molecular gas, or a combination of elemental or molecular gases, supplied from an external source (e.g., a gas line or gas tank). Electrons accelerate toward the discharge electrode 110 and the ionized molecules accelerate toward the opposing electrode 130.

The discharge electrode 110 shape and number determine the shape of the corona discharge 120. The corona treatment system 100 of FIG. 1 including a single needle-shaped discharge electrode 110 creates a conical corona discharge 120 between the tip 112 and the opposing electrode 130.

The corona discharge 120 extends to the opposing electrode 130. An article 140 is shown exposed to (e.g., within) the corona discharge 120 in which the article 140 is exposed to reactive species (e.g., reactive oxygen species (ROS), or reactive nitrogen species (RNS)) generated within the corona discharge 120. The discharge device 105 imparts a surface charge on the article 140 during corona discharge 120 exposure, increasing the surface charge density (σₛ). In some embodiments, the corona treatment system 100 increases σₛ by at least 0.01 μC/m² (e.g., at least 0.1 μC/m², at least 0.2 μC/m², at least 0.5 μC/m², at least 1 μC/m², or at least 5 μC/m²). In some embodiments, the increased article 140 σₛ remains above the pre-CD treatment σₛ for a time period including at least an hour (e.g., at least 2 hours, at least 5 hours, at least 10 hours, at least 15 hours, or at least 24 hours). In various example embodiments, the increased article 140 σₛ remains above the pre-CD treatment σₛ for a time period including at least a day (e.g., at least 2 days, at least 3 days, or at least 4 days). For example, the increased article 140 σₛ remains above the pre-CD treatment σₛ for at least 5 days.

The corona treatment system 100 voltage and electric field generates ionic species (e.g., ions) within the corona discharge 120. In some embodiments, the corona treatment system 100 generates an ion density within the corona discharge 120 of about 0.01×10⁶ ions/cm³ to about 200×10⁶ ions/cm³ (e.g., about 0.1×10⁶ ions/cm³ to about 200×10⁶ ions/cm³, about 1×10⁶ ions/cm³ to about 200×10⁶ ions/cm³, about 10×10⁶ ions/cm³ to about 200×10⁶ ions/cm³, about 50×10⁶ ions/cm³ to about 200×10⁶ ions/cm³, about 100×10⁶ ions/cm³ to about 200×10⁶ ions/cm³, about 150×10⁶ ions/cm³ to about 200×10⁶ ions/cm³, about 0.01×10⁶ ions/cm³ to about 150×10⁶ ions/cm³, about 0.01×10⁶ ions/cm³ to about 100×10⁶ ions/cm³, about 0.01×10⁶ ions/cm³ to about 50×10⁶ ions/cm³, about 0.01×10⁶ ions/cm³ to about 10×10⁶ ions/cm³, about 0.01×10⁶ ions/cm³ to about 1×10⁶ ions/cm³, or about 0.01×10⁶ ions/cm³ to about 0.1×10⁶ ions/cm³).

An article 140 exposed to corona discharge 120 for a treatment time is said to have been exposed to a corona discharge 120 (CD) treatment. The treatment time for a CD treatment can be in a range from 1 s to 60 min (e.g., 10 s to 60 min, 1 min to 60 min, 10 min to 60 min, 30 min to 60 min, 1 s to 30 min, 1 s to 10 min, 1 s to 1 min, or 1 s to 10 s). In an alternative example, the treatment time for a CD treatment can be in a range from 15 s to 10 min (e.g., 30 s to 10 min, 1 min to 30 min, 10 min to 30 min, 20 min to 30 min, 15 s to 20 min, 15 s to 10 min, 15 s to 1 min, or 15 s to 30 s). The article 140 can include a variety of materials and respiratory masks are commonly manufactured from polymer fibers (e.g., a non-woven polypropylene fiber matrix). For instance, non-limiting examples may include metals, dielectric, polymer, plastic, or glass, or combinations thereof.

In some embodiments, the corona treatment system 100 increases the filtration efficiency to an established level (e.g., filtration efficiency as measured by NIOSH standardized testing guidelines such as 42 CFR 84, or USFDA good manufacturing practice (GMP) regulations 21 CFR sections 210, 211 and 820) for materials and respiratory masks (e.g., an NaCl polydispersed test aerosol with a median particle diameter of 0.075 μm, a mass mean diameter of 0.26 μm, and a geometric standard deviation of 1.83 discharged at a flow rate of 85 liters per minute). In some embodiments, the CD treatment can increase the filtration efficiency to at least 40% (e.g., at least 80%, or to at least 95%). In some embodiments, the CD treatment can increase the filtration efficiency by at least 1% (e.g., at least 2%, at least 5%, at least 10%, at least 20%).

In some embodiments, the article 140 is exposed to corona discharge 120 for more than one treatment time. In such embodiments, a storage time in which the article 140 is not exposed to the corona discharge 120 separates each treatment time. A treatment time and a storage time can be termed a ‘cycle.’ The storage time can be in a range from 1 s to 60 min (e.g., 10 s to 60 min, 1 min to 60 min, 10 min to 60 min, 30 min to 60 min, 1 s to 30 min, 1 s to 10 min, 1 s to 1 min, or 1 s to 10 s). The article 140 can undergo one or more cycles, such as a range from 2 cycles to 10 cycles (e.g., 4 cycles to 10 cycles, 6 cycles to 10 cycles, 8 cycles to 10 cycles, 2 cycles to 8 cycles, 2 cycles to 6 cycles, or 2 cycles to 4 cycles).

In some embodiments, the corona treatment system 100 includes relative motion between the opposing electrode 130 and the discharge electrode 110 such that the corona discharge 120 passes over an area of the opposing electrode 130 for a time. For example, the opposing electrode 130 can be a moving belt having more than one article 140 disposed on the belt surface. In such an example, relative motion is created by the belt and discharge electrode 110, either by the corona treatment system 100 or by an external belt power source, wherein the articles 140 are exposed to the corona discharge 120 while in communal motion with the belt surface. In some embodiments, relative motion is created between the article 140 and discharge electrode 110 through motion of the discharge electrode 110 with respect to the article 140.

FIG. 1D depicts a top down view of the opposing electrode 130 and the corona discharge 120 cross-section at the opposing electrode 130 surface. The area of the opposing electrode 130 which the corona discharge 120 covers can be considered a treatment area 122. In general, this treatment area 122 can be of any shape but some discharge electrodes 110, e.g., needles, can produce a circular treatment area 122. For example, a discharge electrode 110 in the shape of a wire can create a treatment area 122 that is symmetric about the longitudinal axis of the wire discharge electrode 110 and runs the length of the wire discharge electrode 110. In some embodiments, the wire discharge electrode 110 can be from about 1 cm to about 50 cm or more (e.g., from about 1 cm to about 50 cm, from about 20 cm to about 50 cm, from about 40 cm to about 50 cm, from about 1 cm to about 30 cm, from about 1 cm to about 10 cm).

Other shapes and sizes of the treatment area 122 can be achieved by varying the shape and number of discharge electrodes 110 and the voltage. The treatment area 122 covers at least a portion of the opposing electrode 130 and the article 140, and can be larger than the article 140.

FIG. 1E depicts a chart displaying a transverse view of a simulated corona discharge 120 of a discharge electrode 110 in the shape of a wire. The y-axis of the chart shows a vertical range of about 3 cm and the discharge electrode 110 wire at a height of about 2.5 cm. The simulated corona discharge 120 shows a treatment area 122 width of about 7 cm. Without wishing to be bound by theory, because the depicted discharge electrode 110 is a wire, the treatment area 122 can be considered symmetric about the discharge electrode 110 wire and extending coaxially for the length of the discharge electrode 110 wire.

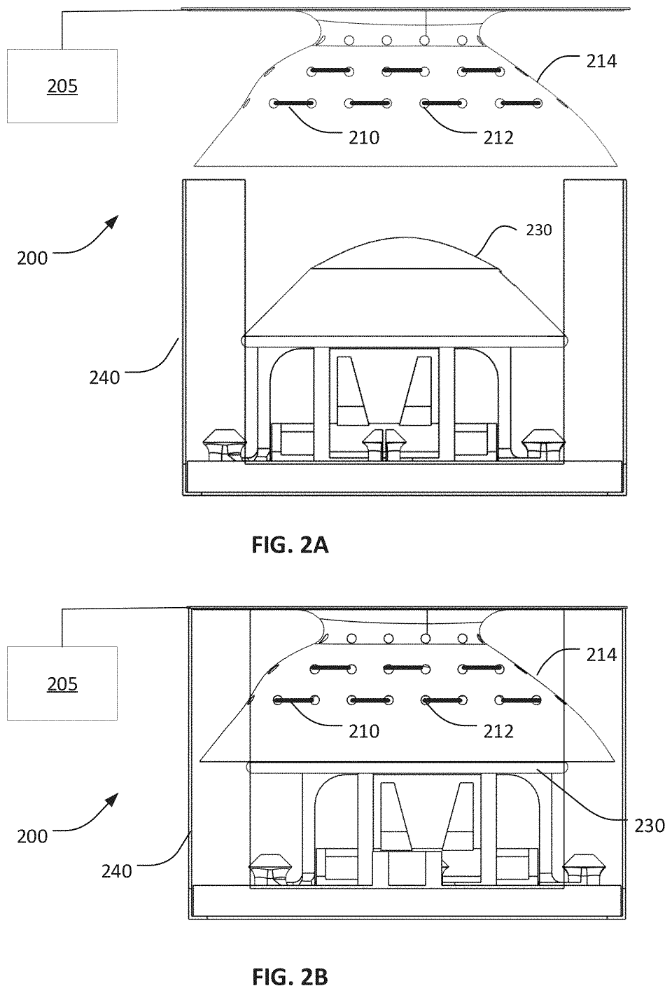

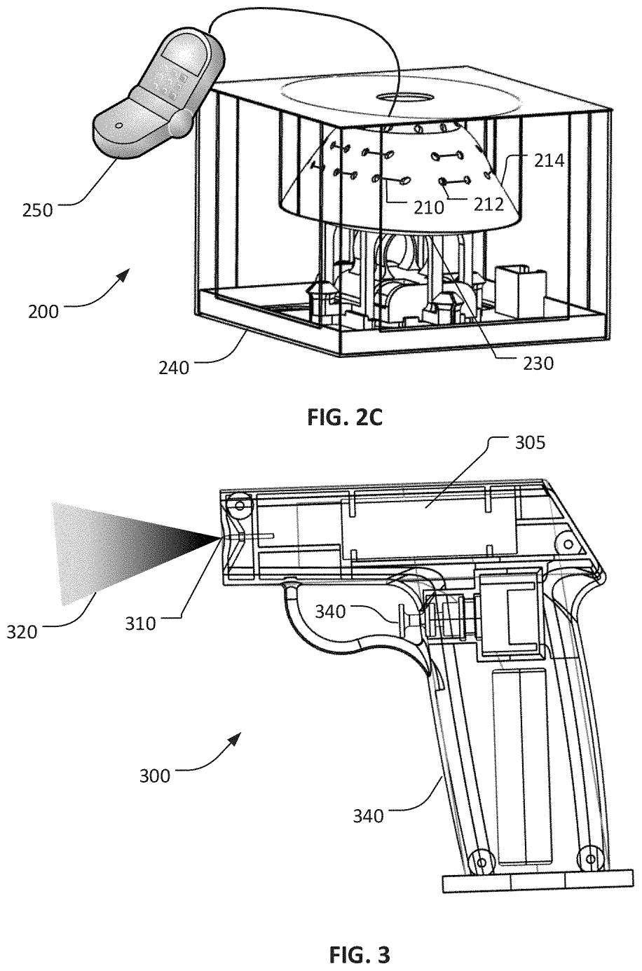

Exemplary embodiments of a corona treatment system 100 are shown in FIGS. 2A to 2D. FIG. 2A is a schematic diagram of corona treatment system 200 depicting an inverted bowl-shaped fixture 214 including a wire-shaped discharge electrode 210. The fixture 214 is positioned over an opposing electrode 230 shaped to the inner surface of the fixture 214. The discharge electrode 210 is in electrical communication with a discharge device 205 sustaining the voltage of the discharge electrode 210. In some embodiments, the fixture 214, housing 240, and/or the opposing electrode 230 include a mechanism for releasably securing an article 140, such as a respirator mask. In some embodiments, the discharge electrode 110 is a wire-shaped article 140 in the form of a ring. In such embodiments, the corona treatment system 100 includes an electrode support structure (e.g., fixture 214) configured to receive and position the wire by the distance (d) (e.g., the electrode-sample distance) from the article 140.

The fixture 214 includes holes 212 through which the discharge electrode 210 is woven, such that the discharge electrode 210 is intermittently positioned on the inner- or outer-surface of the fixture 214.

The corona treatment system 200 includes an opposing electrode 230 partially encased within a housing 240. The housing 240 is rectangular with a height approximately equal to a width and length. In some embodiments, the height, width, and length, can be in a range from about 10 cm to about 50 cm (e.g., about 20 cm to about 50 cm, about 30 cm to about 50 cm, about 40 cm to about 50 cm, about 10 cm to about 40 cm, about 10 cm to about 30 cm, or about 10 cm to about 20 cm). In some embodiments, the housing 240 includes walls on the exterior surfaces which when sealed form a gas- or liquid-tight chamber and can include one or more ports for introducing non-atmospheric gases.

FIG. 2B depicts the fixture 214 positioned above the opposing electrode 230 such that a planar surface forming the top surface of the fixture 214 abuts the housing 240 on the upper edge of all four corners. In embodiments in which the housing 240 includes walls, the planar surface can complete the gas- or liquid-tight seal. The housing 240 inner volume, defined by the four walls and floor, is at least 1,000 cm³ (e.g., at least 10,000 cm³, or at least 100,000 cm³).

The inner and outer surfaces of the fixture 214 and opposing electrode 230, respectively, are formed such that when positioned adjacent, the surfaces are separated by an approximately equal distance such that the corona discharge 120 generated between the surfaces is approximately uniform across the opposing electrode 230 outer surface. The outer surface of a mask disposed on the opposing electrode 230 outer surface is thereby exposed to the approximately uniform corona discharge 120 emitted by the discharge electrode 210 on the fixture 214 inner surfaces.

FIG. 2C is a perspective view of an alternative corona treatment system 200 including a hand-held controller 250 for portability and user interaction. The housing 240 (e.g., treatment controller) can be optionally connected to the controller 250 by a wire or the housing 240 can be integrated with the controller 250. The controller 250 includes a user interface (e.g., buttons, dials, display, or touch screen) through which a user can control the corona treatment system 200 and, optionally, can include two-way electronic communication transceivers for wired (e.g., USB, or Lightning) or wireless (e.g., Bluetooth®, NFC, or Wi-Fi) communication with local or dispersed networks. The control device 150 may be operated with a power source that provides energy at a range of voltages, e.g., between 5 and 240 V. In some embodiments, the energy source can be one or more batteries, e.g., alkaline batteries, lithium-ion batteries. In some embodiments, the energy source for the control device 150 can be a cord that is then affixed to a standard outlet operating a local voltage, e.g., 120 V, 240 V. The controller 250 can include a high voltage transformer to increase the voltage of the energy source to the range required for corona discharge, e.g., 10-100 kV.

The user can directly input CD treatment parameters such as voltage, current, discharge electrode-article distance, or treatment time for CD treatment of an article 140. In some embodiments, the controller 250 can operate more than one corona treatment system 200. In various alternative embodiments, the controller 250 is partially or completely embedded in the housing 240.

FIG. 3 is a schematic illustration of an alternative corona treatment system 300 in which the discharge electrode 310 and corona discharge device 305 are enclosed in a trigger-operated, hand-held casing 340 including a handle. The casing 340 of FIG. 3 allows increased portability and simplified operation for applying CD treatment to a variety of articles 140. Some or all of the components of the corona discharge device 305 are enclosed in the casing 340, including electrical connections, transformers, power supplies, or control electronics. In some embodiments, the casing 340 includes an electrical connection for operation with an external power supply (e.g., a wall outlet). In some embodiments, the casing 340 is sized to fit in a hand of a user. For example, non-limiting sizes of the handheld device may be less than 10 inches in one dimension, less than 4 inches in a second, and less than 4 inches in a third dimension.

In this embodiment, the corona discharge 320 is generated by one or more discharge electrode 310 integrated with the casing 340 in which the corona discharge 320 emitting surface (e.g., the tip or wire) extends from the casing 340 in which the discharge electrode 310 is housed within a recessed cavity of the casing 340 (e.g., a nozzle) which a user orients toward an article 140. A user operates the corona treatment system 300 with a hand and orients the discharge electrode 310 (e.g., the nozzle) before operating the discharge mechanism 342 causing the corona discharge device 305 to generate a corona discharge 320. The user directs the corona discharge 320 a surface, or article 140, for CD treatment. FIG. 3 depicts the corona discharge device 305 integrated internally within the casing 340 but the corona discharge device 305 can be integrated externally. The corona discharge device 305 integrated with the corona treatment system 300 can be operated at any voltage described herein.

Quantification of the microbial population reduction can include the determination of a biologic log reduction. A biologic log reduction is the reduction of detectable biologic markers (e.g., bacteria or viruses) by a logarithmic power value, for example, a log reduction of 1 corresponding to a reduction of detectable biologic markers by a factor of 10 (10⁻¹), or a log reduction of 2 corresponding to a reduction by a factor of 100 (10⁻²). In some embodiments, the log reduction can be in a range from 1 to 6 (e.g., 2 to 6, 3 to 6, 4 to 6, 5 to 6, 1 to 5, 1 to 4, 1 to 3, or 1 to 2).

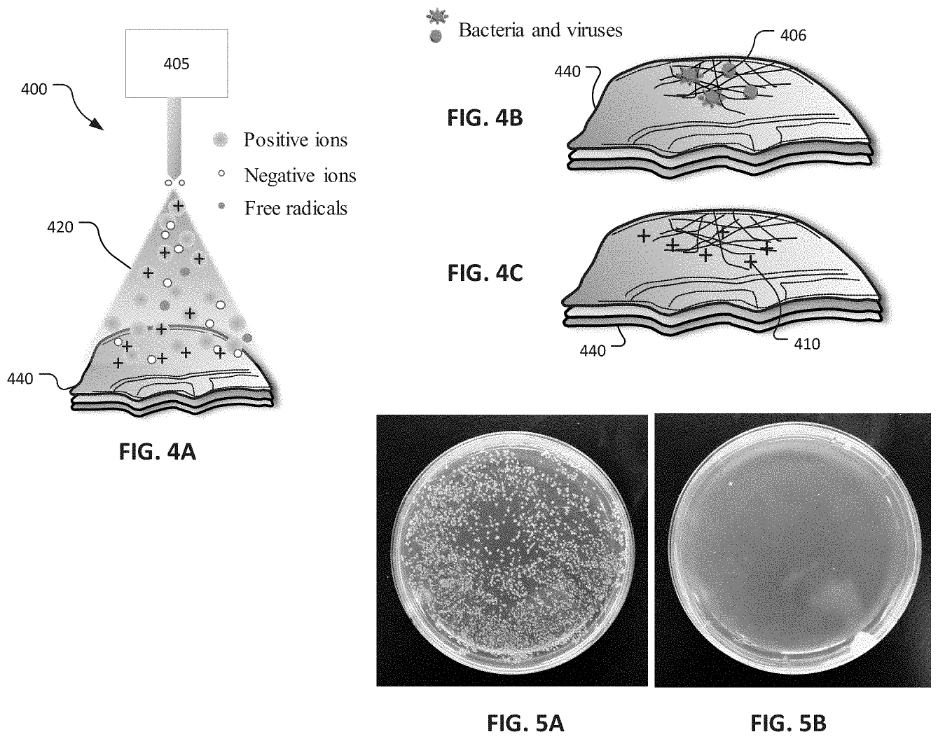

FIGS. 4A to 4C depict an exemplary process for using the corona treatment system 400 to disinfect an article 440. In FIG. 4A an article 440 is exposed to the corona discharge 420 of a corona discharge device 405.

FIG. 4B depicts an exemplary contaminated article 440 before processing with a corona treatment system 400 (not shown). In general, the contaminated article 440 can be contaminated with a biological microorganism 406. In some embodiments, the biological microorganism 406 can be virus or bacteria. In some embodiments, the bacteria can be E. coli. In some embodiments, the virus can be SARS-COV-2.

FIG. 4C depicts the contaminated article 440 of FIG. 4B after processing with a corona treatment system 400 (not shown) to become a disinfected article 440. In the process of corona treatment, the biological microorganisms 406 have been deactivated or rendered non-viable. In addition to treating the disinfected article 440, the corona treatment system 400 (not shown) can inject a charge into the material of the disinfected article 440 through exposure to the corona discharge 420.

An article that has been subjected to CD treatment includes viable microbial population (e.g., remains nonsterile) which can be removed and quantified via microbiological assay such as agar plate growth and colony counting. For example, FIG. 5A depicts a culture of a sample of E. coli from an article 140 that was not processed with the corona treatment system 100. Each visual white dot represents the presence of an individual E. coli colony. FIG. 5B depicts a culture of a sample of E. coli from an article 140 that was processed with the corona treatment system 100 at a discharge electrode 110 potential of 20 kV for 7.5 min. There are no apparent white dots corresponding to E. coli colonies. These assays can indicate a log reduction of 3 (10⁻³) can be achieved with CD treatment. In general, extended treatment time and higher discharge electrode 110 voltage can lead to an log reductions of 2 or more (e.g., 3 or more, 4 or more, 5 or more, or 6 or more).

In some embodiments, quantification of the microbial population reduction can include the determination of a sterility assurance level (SAL). A SAL is the probability that an article that has been subjected to CD treatment includes viable microbial population. These results in FIGS. 5A and 5B indicate an SAL of 10⁻³ can be achieved with these CD treatment parameters. In general, extended corona treatment system 100 treatment time and higher discharge electrode 110 voltage can lead to an SAL lower than 10⁻³ (e.g., lower than 10⁻³, lower than 10⁻⁴, or lower than 10⁻⁵). In some embodiments, the SAL can be lower than 10′.

### EXAMPLES

### Example 1: Disinfection and Electrostatic Recovery of N95 Masks by Corona Discharge

The corona discharge (CD) disinfection of N95 masks using E. coli, P. pastoris, and Geobacillus as a model organism indicates that CD achieves a log reduction of 3 within 7.5 min at 25 kV voltage on E. coli with a single treatment, consuming at least 1.25 W of power. A multi-cycle treatment increases reductions. These treatments recover the electrostatic surface charge of N95 masks. Ion component analyses indicate antimicrobial reactive nitrogen species (RNS) and reactive oxygen species (ROS) generated by CD improve disinfection efficiency by causing damage to pathogenic DNA and proteins.

### Experimental Conditions

Corona Discharge.

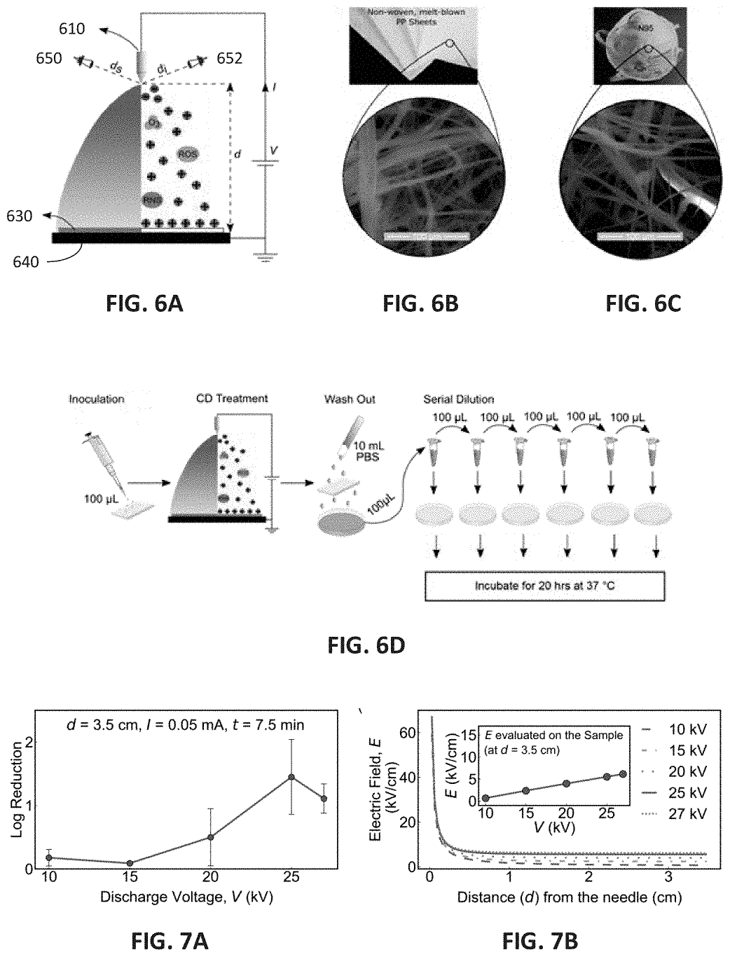

The corona discharge system is shown in FIG. 6A. The voltage and current applied to the discharge electrode 610 were controlled by a high-voltage DC power supply (XP Glassman Co. Ltd, FJ Series, 120 W) with a controllable voltage range of 0 to ±60 kV and current range of 0 to 0.20 mA, respectively. The electrode 610 is composed of tungsten and curvature of the electrode 610 tip was 1000 μm compared to 120 μm for the 5 cm tungsten wire. The polarity of the discharge electrode 610 was set to be positive (e.g., an anode). A sample 630 was mounted on a stainless-steel stage 640 ground electrode (e.g., cathode). The electrode 610 is arranged vertically above the sample 630 at a controllable distance (d), oriented coaxially with the middle of the sample 630. CD was applied at atmospheric pressure in an ambient gas environment (e.g., air).

All experiments were conducted with discharge current (I) set to at least 0.05 mA. The effect of treatment time (t) was determined by treating the sample 630 by time periods varying from 30 s to 15 min with voltage (V) set to 25 kV and distance (d) to 3.5 cm. The effect of distance (d) was determined by treating the sample 630 with voltage (V) set to 25 kV and d varied from 2 cm to 5 cm. The effect of voltage (V) was determined analyzed by treating the sample 630 with distance (d) to 3.5 cm and varying V from 10 kV to 27 kV.

Characterization of Corona Discharge.

A spectrometer 650 (such as an AvaSpec-ULS2048CL-EVO manufactured by Avantes) acquired the CD spectrum. A fiber optic cable of spectrometer 650 was positioned at a distance (ds) of 7.5 cm away from the electrode 610 tip (see FIG. 6A). The ion density from the discharge was measured using an air ion counter 652 (such as an AIC2 manufactured by Alpha Lab Inc.). The electrode 610 was operated as an anode (e.g., positive polarity), and the spectrometer 650 was positioned at a parallel distance (di) of 7.5 cm away from the electrode 610 tip. Ozone generated due to the combination of molecular and atomic oxygen was measured using an ozone meter (such as ozone meter model FD-90A-O3 manufactured by Forensics Detectors).

Electric Field Simulation.

COMSOL simulation was performed to calculate the electric potential distribution in the CD controlled by different parameters. A one dimensional model was setup using a point to plane configuration. The electron and ions continuity and momentum equation is solved using the drift-diffusion equation coupled with Poisson's equation. The electrostatic potential V is computed with Equation (2).

−∇·ε₀εᵣ∇V=ρ (2)

where, ρ is the space charge density and ε₀, εᵣ are permittivity in free space and relative permittivity, respectively.

Disinfection Effect Evaluation.

Six 5 cm×5 cm sample 630 of non-woven, melt-blown polypropylene (PP) fabric (total of 1.8 mm thickness) were inoculated with Escherichia coli (E. coli) (e.g., a DH5-α E. coli derivative, purchased from New England Biolabs Inc.) to evaluate CD disinfection effectiveness. E. coli cell cultures were prepared using 100 mL of lysogeny broth (LB) and incubated overnight at 37° C. for each experiment. The cell pellets were collected at 2500 rpm for 10 min and washed one time with 1 mL of phosphate buffer (PBS, pH=7.4) to obtain an initial concentration of 10¹⁰ CFU/mL.

For Pichia pastoris (a species of methylotrophic yeast), cell cultures were prepared using 5 mL of Yeast Extract-Peptone-Dextrose (YPD) broth with 20% glucose and incubated overnight at 30° C. at 300 rpm to achieve an initial concentration of 10⁸ CFU/mL). Geobacillus (which produces heat resistant spores) cells were prepared using 100 mL of nutrient broth (Difco™ nutrient broth for cultivation of nonfastidious microorganisms) and incubated overnight at 50° C. at 120 rpm (reaching an initial concentration of 10⁴ CFU/mL).

For each experiment, 100 μL of the prepared culture was disposed on the sample 630 and spread to form a uniform layer covering the sample 630 surface area and mounted on the stage 640. The sample 630 was exposed to CD based on the parameters mentioned above. The sample 630 was transferred to a petri dish and surviving biologic colonies were washed out using 10 mL of PBS. 100 μL of inoculated PBS solution was added to 900 μL of PBS and this process repeated to acquire six serial dilutions. 100 μL of each dilution was deposited onto a respective agar plate for incubation at 37° C. After 20 hours, surviving colonies were counted. The log reduction is given by Equation (1).

log reduction=log(initial colonies)−log(surviving colonies) (1)

All experiments were conducted in triplicate.

Expression and Purification of sfGFP.

The arabinose-inducible plasmid pBad-sfGFP for sfGFP expression was purchased from Addgene. The sfGFP gene was codon-optimized for E. coli with a C-terminal 6-His affinity tag. DH10B E. coli cells transformed with pBad-sfGFP were used to inoculate 500 mL of LB medium containing 100 μg/mL Ampicillin. After the E. coli in the LB medium culture grown to an optical density at 600 nm (OD600) of 0.1 with shaking at 37° C., 0.1% Arabinose was added to induce the sfGFP expression. Solutions were shaken for 40 h at 37° C. and 500 mL of cell culture collected by centrifugation.

The protein was purified using TALON® metal affinity resin (such as those manufactured by TaKaRa Bio.). The cell pellet was resuspended by ultrasonic cell disruptor in Lysis buffer (50 mM PBS, 300 mM sodium chloride, pH 7.4). The supernatant was disposed in 1 mL TALON® resin and bound for 30 min. The bound resin was washed with 50 volumes of Lysis buffer. Protein was eluted from the bound resin with 5 mL volumes of elution buffer (50 mM PBS, 300 mM sodium chloride, 300 mM imidazole, pH 7.4) and concentrated to 1 mL.

Plasmid DNA Culture and Purification.

The plasmid pPIC9K-A2aR is purified using plasmid purification kit purchased from Agilent.

SDS-PAGE

Prepare the separation gel (12%).

Prepare the stacking gel (12%). Incubate sample 630 with loading buffer (TriTrack DNA Loading Dye (6×), Thermo Fisher Scientific) for 20 min at room temperature. Load sample and molecular mass protein markers into wells for separation by electrophoresis after the gel polymerized. Run at 120 V for 70 min.

DNA Gel Electrophoresis.

Samples were loaded into wells of a 1% agarose gel (purchased from Bio-Rad) made with 1×TAE buffer. Samples included 5 μL of plasmid with different treatment time and 5 μL of a nucleic ladder. The gel was ran at 100 V in an agarose gel chamber filled with 1×TAE buffer for 40 minutes.

Fluorescence Measurement.

The treated sfGFP samples were transferred to the wells of a fluorescence-compatible 96 well plate to a total well volume of 150 μL. sfGFP fluorescence measurement were taken using an excitation wavelength of 483 nm and an emission wavelength of 535 nm using a Bio Synergy 2 Multi-Detection Microplate Reader. Each measurement was repeated in triplicate with elution buffer as a blank.

Recharge Effect Evaluation.

Commercially available N95 respirators (such as model 3M-8210 manufactured by 3M) were exposed to CD with V=25 kV, I=0.05 mA and d=3.5 cm. Samples 630 were handled to prevent environmental charge accumulation. After CD treatment, the surface potential of the top layer of N95 respirators was monitored at 3 different locations using an electrostatic field meter (model FMSX-004 manufactured by SIMCO Ion). The measurements were taken 25 mm from the sample 630 surface at constant intervals starting from 30 minutes to 9 days after treatment. The surface charge density (σₛ) was calculated by solving Equation (2). In addition, the sample 630 was stored in a humidity controlled chamber (model FSDCBLK100b manufactured by FORSPARK) where the relative humidity was maintained between 45% and 60% over the period of observation. The experiments were repeated three times using new control sample 630 in pristine condition each time.

Filtration Efficiency Test.

Samples 630 were subjected to a 1 minute loading test derived from a standard testing procedure from the National Institute for Occupational Safety and Health (NIOSH) procedure number TEB-APR-STP-005976 using TSI® CERTITEST® Model 8130 Automated Filter Tester manufactured by Nelson Labs where it was preconditioned for 25 hours±1 hour, at a relative humidity of 85±5% and a temperature of 38° C.±2.5° C. Samples 630 were tested for a 1 min load test using a NaCl solution at 85±4 L/min flow rate. The solution particle size was 0.078±0.020 μm and the pressure drop across the FFR at this flow rate was also monitored. The samples 630 were secured on the edges to prevent leakage.

Results and Discussion

Experimental Setup.

FIG. 6A is a schematic diagram of the corona discharge (CD) application setup with respect to a sample 630. Samples 630 were exposed to the CD by an electrode 610 maintained at a voltage (V). Samples 630 were layered non-woven polypropylene (PP) fabrics, as described above. Six layers of PP fabrics were stacked together for the CD testing to reach a total thickness of 1.8 mm. SEM images were taken was conducted to compare sample 630 microstructure with N95 masks (model 07048 manufactured by 3M). FIGS. 6B and 6C are SEM images of layered non-woven PP fabrics, and filter layer of N95 masks, respectively. As shown in FIGS. 6B and 6C, similar fiber diameter, density, and porosity is demonstrated.

Systematic disinfection tests were conducted to evaluate the disinfection efficacy of CD. E. coli was used in the disinfection tests unless otherwise noted. FIG. 6D schematically illustrates the disinfection experiments procedure including inoculating controllable amount of E. coli on the left, CD treatment in the second image, wash out surviving colonies in the third image, and serial dilution using PBS, plating and incubation in the right-most series of images. As depicted in FIG. 6D, specimens were inoculated with 100 μL of E. coli solution. Samples 630 were exposed to CD based on multiple parameters, including corona voltage (V), treatment time (t), distance between the sample and the discharge electrode (d), electrode geometry (needle vs. wire), number of treatment cycles (n) and storage time between each cycle (s). Surviving colonies were washed out with phosphate buffer (PBS), followed by serial dilution, culturing and CFU counting. Log reduction was calculated using Equation (1), described above.

Effect of Discharge Voltage (V).

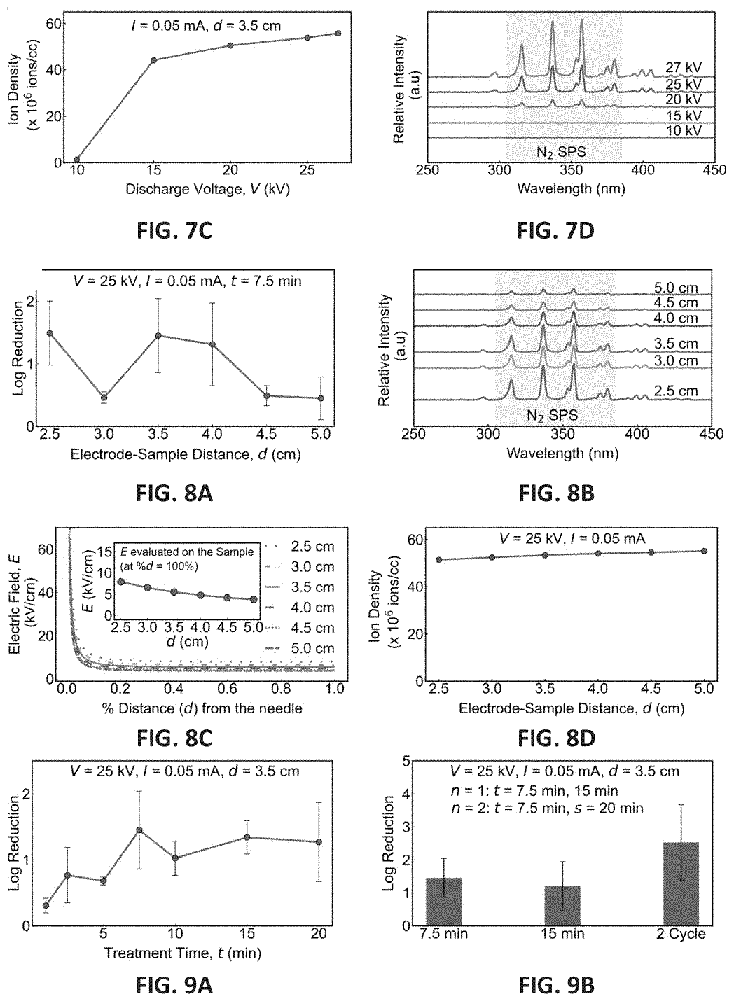

FIG. 7A is a line graph comparing electrode 610 discharge voltage Von the x-axis with logarithmic reduction of detected E. coli on the y-axis. Discharge parameters are noted inset at the top of the chart. The CD parameters of FIG. 7A were I=0.05 mA and d=3.5 cm. As shown in FIG. 7A, CD treatment electrode 610 voltage of greater than 15 kV achieves a disinfection effect. The log reduction increases with the increase of voltage, reaching 0.5±0.45 at V=20 kV and 1.45±0.59 at V=25 kV as average values for treatment time of t=7.5 min and electrode 610 sample 630 distance of d=3.5 cm.

However, the disinfection effect reduces at V=27 kV. Arcing was observed during the treatment, reducing the area being exposed to CD. The increased disinfection efficiency is explained by increased electric field, ion density, and reactive oxygen and nitrogen species (ROS, RNS). Electric fields greater than 5 kV/cm generate a voltage of 1V across cell membranes leading to electric breakdown of the cell walls due to electroporation, see at least Zimmermann, J. et al., titled Test For Bacterial Resistance Build-Up Against Plasma Treatment, New Journal of Physics 2012, 14 (7), 073037 which is incorporated herein by reference in its entirety.

FIG. 7B is a line chart showing simulated electric field (E) on the y-axis compared to distance between the electrode 610 and sample 630 on the x-axis for five electrode 610 voltages. The electric field was simulated using COMSOL software. Inset to FIG. 7B is a second line chart showing measured electric field (E) on the y-axis compared to electrode 610 voltage (V) on the x-axis. As V increases, E on the sample increases.

As shown in FIG. 7B, simulated the electric field (E) on the sample 630 surface increases with CD electrode 610 voltage, to a maximum of E=3.96 kV/cm for V=20 kV and E=5.52 kV/cm for V=25 kV showing CD creates electric fields across the microorganisms to deactivate them.

High densities of positive and negative ions (e.g., in a range from 5×10⁴ and 5×10⁶ ions/cm³) can cause mechanical damage to cell envelopes, see at least Noyce, J. et al, titled Bactericidal Effects Of Negative And Positive Ions Generated In Nitrogen On Escherichia coli, Journal of electrostatics 2002, 54 (2), 179-187 which is incorporated herein by reference in its entirety.

FIG. 7C is a line chart showing ion density on the y-axis compared to electrode 610 voltage on the x-axis. As shown in FIG. 7C, ion density increases with the increase of the CD electrode 610 voltage. When the voltage was increased to 25 kV, the ion density can reach as high as 53.87×10⁶ ions/cm³, effective for disinfection.

FIG. 7D is a line chart comparing the spectrum intensity on the y-axis generated by CD treatment at five voltages to wavelength on the x-axis detected by an optical emission spectrometer (OES) described above. The highlighted area in the wavelength range between 300 nm and 380 nm correspond to N₂ SPS peaks.

The intensity of RNS corresponds with the result of FIGS. 7A and 7B, with negligible RNS species observed at voltages V=15 kV or below and increasing intensity with voltages from V=20 kV and higher. ROS and RNS generated by CD, include assemblies of O₃, O₂, O., H₂O₂, NO, NO₂, HNO₃, HNO₂, ONOO⁻ and OH⁻ which cause oxidative damage in molecular targets leading to DNA, protein, and lipid breakdowns leading to cell death, see at least Dobrynin, D. et al., titled Physical and Biological Mechanisms of Direct Plasma Interaction with Living Tissue, New Journal of Physics 2009, 11 (11), 115020-50 which is incorporated herein by reference in its entirety.

The peaks detected in this experiment correspond with wavelength at 315.93 nm, 337.13 nm, and 357.69 nm which correspond to nitrogen second positive system (N₂ SPS), confirming the presence of RNS. ROS have also been generated but are difficult to detect having a short half-life losing most energy colliding with other particles. However, ozone (O₃) having a half-life measured in hours, 0.1 ppm of O₃ was measured using an ozone detector after t=7.5 min. Thus, electric field, ion density, and reactive species (RNS and ROS) increase with increased electrode 610 voltage, and contribute to CD treatment disinfection effect.

Effect of Electrode-Sample Distance.

FIG. 8A is a line graph comparing electrode 610 discharge voltage Von the x-axis with logarithmic reduction of detected E. coli on the y-axis. Discharge parameters are noted inset at the top of the chart, discharge parameters being I=0.05 mA and V=25 kV. As shown in FIG. 8A, the electrode 610-sample 630 distance d (shown in FIG. 6A) in a range between 3 cm and 4.5 cm achieves CD treatment disinfection effect.

At distances of d of less than or equal to 3 cm, increased electric arcing led to physical damage to the samples 630, and a log reduction of 1.49±0.51. The log reduction increased from 0.46±0.09 for d=3 cm to 1.45±0.59 and 1.31±0.66 for d=3.5 and 4 cm, respectively. In a range between d=3.5 and 4 cm, the CD treatment covers the entire 5 cm×5 cm sample with a strong diffuse cone leading to increased disinfection effect.

At distances (d) of greater than or equal to 4.5 cm, the CD treatment zone is reduced, reducing disinfection effect corresponding with reduced relative intensity of N₂ SPS peaks with the increase of d in FIG. 8B, indicating very weak diffuse cone at distances (d) greater than or equal to 4.5 cm. The results match with Warburg's law which states the treatment area of the CD treatment zone (e.g., the diffuse cone) decreases at shorter distances, while the reactive particles (ROS, RNS and ions) become weaker at larger distances, see at least Scholtz, V. et al., The Microbicidal Effect of Low-Temperature Plasma Generated by Corona Discharge: Comparison of Various Microorganisms on an Agar Surface or in Aqueous Suspension. Plasma Processes and Polymers 2010, 7 (3-4), 237-243, which is incorporated herein by reference in its entirety.

FIG. 8C is a line chart showing simulated electric field (E) on the y-axis compared to normalized distance between the electrode 610 and sample 630 on the x-axis for six electrode 610 distances (e.g., 2.5 cm, 3.0 cm, 3.5 cm, 4.0 cm, 4.5 cm, and 5.0 cm). The electric field was simulated using COMSOL software. Inset to FIG. 8C is a second line chart showing measured electric field (E) on the y-axis compared to electrode 610 distance (d) on the x-axis. As d increases, E on the sample decreases.

As simulated in FIG. 8C, the electric field E applied to the sample 630 reached the highest value of E=7.94 kV/cm at a distance d of 2.5 cm. With increased distance, the electric field reduces to E=6.52 kV/cm and E=5.52 kV/cm for d=3 cm and d=3.5 cm respectively before dropping below 5 kV/cm for d greater than or equal to 4 cm. This trend also matches with the observed log reduction of FIG. 8A.

FIG. 8D is a line chart showing ion density on the y-axis compared to distance between the electrode 610 and sample 630 on the x-axis. Discharge parameters are noted inset at the top of the chart. As shown in FIG. 8D, ion density increases with the increase of the CD electrode 610 voltage. The ion density remains relatively constant with increasing distance as shown in FIG. 8D. The results correspond with increased disinfection effect due to high electric field values and reactive species (RNS and ROS). An electrode-sample distance (d) in a range between 3.5 cm to 4 cm achieves highest disinfection efficacy of up to log reduction of 1.45±0.59 against E. coli.

Effect of Treatment Time.

FIG. 9A is a line graph comparing treatment time in minutes on the x-axis with logarithmic reduction of detected E. coli on the y-axis. Discharge parameters are noted inset at the top of the chart, discharge parameters being I=0.05 mA, V=25 kV, and electrode-sample distance (d) of 3.5 cm. The treatment time (t)(e.g., time sample 630 was exposed to CD) was varied from 1 min to 20 min. Higher values of treatment time corresponds to increases in log reduction of E. coli population. The disinfection effect increases from 0.34±0.10 log reduction for t=1 min to 1.56±0.20 log reduction for t=7.5 min as shown in FIG. 9A. The disinfection efficacy cannot be significantly increase by further increasing the treatment time to 20 min. Therefore, treatment time of 7.5 min is effective.

Effect of Cycle Time and Storage Time.

Samples 630 were exposed to multiple treatment cycles (n) with a storage time (s) between each cycle to allow E. coli to completely react to the applied CD see at least Han, L. et al., titled Mechanisms Of Inactivation By High-Voltage Atmospheric Cold Plasma Differ For Escherichia Coli And Staphylococcus Aureus. Applied and environmental microbiology 2016, 82 (2), 450-458.37, 52, which is incorporated herein by reference in its entirety.

FIG. 9B is a bar chart comparing logarithmic reduction of detected E. coli on the y-axis to treatment times on the x-axis. Discharge parameters are noted inset at the top of the chart, set to V=25 kV, I=0.05 mA and d=3.5 cm. Treatment times of t=7.5 mins and storage time of s=20 min for n=2 cycles, the log reduction increased to 2.52±1.14 in comparison with the 1.206±0.33 achieved with t=15 mins of continuous treatment as shown in FIG. 9B.

Adding storage time between each cycle of treatment enhances the disinfection effect, and storage time greater than 5 min increases the disinfection effect. The reactive species remain active during storage time, continuing to disrupt cell membranes between cycles cycle, see at least Ziuzina, D. et al., titled Atmospheric Cold Plasma Inactivation of Escherichia Coli in Liquid Media inside a Sealed Package. Journal of applied microbiology 2013, 114 (3), 778-787, which is incorporated herein by reference in its entirety.

Effect of Electrode Geometry.

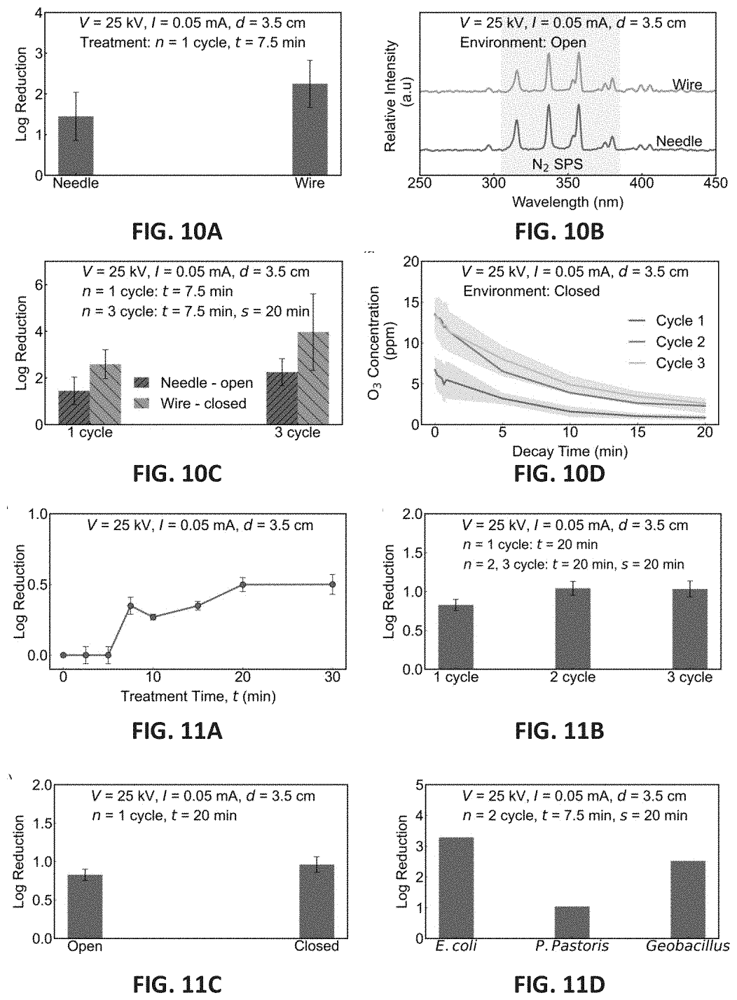

The discharge electrode 610 shape influences the CD initiation voltage (e.g., the voltage at which CD emits from the electrode 610). FIG. 10A is a bar chart comparing the log reduction in E. coli populations on the y-axis to two electrode 610 shapes (e.g., needle and wire) on the x-axis. Discharge parameters are noted inset at the top of the chart, discharge parameters being I=0.05 mA, d=3.5 cm, n=1, t=7.5 min, and V=25 kV. A tungsten wire electrode 610 (120 μm diameter, 5 cm length) was used to study the disinfection efficacy against E. coli, the log reduction increases to 2.25±0.57 compared to 1.45±0.59 achieved with the tungsten needle.

FIG. 10B is a line chart comparing the spectrum intensity on the y-axis generated by CD treatment using two needle shapes to wavelength on the x-axis detected by an optical emission spectrometer (OES) described above. The highlighted area in the wavelength range between 300 nm and 380 nm correspond to N₂ SPS peaks. Discharge parameters are noted inset at the top of the chart and are the same as FIG. 10A.

The relative intensity of N₂ SPS peaks observed on the needle-shaped and wire-shaped electrode 610 were comparable but longer discharge surface (e.g., 5 cm length) of the wire-shaped electrode 610 exposed the entire 5 cm×5 cm surface area of the sample 630 to a uniform CD treatment zone.

The sample enclosed in a casing (e.g., a closed environment) and treated for n=3 cycles. FIG. 10C is a bar chart comparing the log reduction in E. coli populations on the y-axis to two cycle counts on the x-axis. Two electrode 610 shapes (e.g., needle-shaped and wire-shaped) were used, the needle-shaped electrode 610 being in an open environment and wire-shaped electrode 610 being in an enclosed environment. Discharge parameters are noted inset at the top of the chart, discharge parameters being I=0.05 mA, d=3.5 cm, t=7.5 min, and V=25 kV. The left bars represent n=1, and the left bars represent n=3 with s=20 min.

FIG. 10C shows the log reduction increases to 3.96±1.64 using tungsten wire-shaped electrode 610 compared to 2.59±0.62 using needle-shaped electrode 610.

FIG. 10D is a line chart comparing ozone concentration in ppm on the y-axis to decay time (min) on the x-axis) for one, two, and three treatment cycles. Discharge parameters are noted inset at the top of the chart, discharge parameters being I=0.05 mA, d=3.5 cm, t=7.5 min, and V=25 kV. Slower ozone decay inside the closed environment after each treatment cycle (shown in FIG. 5d) ensured consistent exposure to ozone during the treatment phase and storage phase and corresponds to increased log reduction.

Disinfection Efficacy Against Yeast and Spores.

Disinfection experiments on Pichia pastoris (a species of methylotrophic yeast) and Geobacillus (which produces heat resistant spores) were used to test broad-spectrum disinfection efficacy of CD. Pichia pastoris and Geobacillus include thicker cell walls than E. coli having increased tolerance against membrane rupture and DNA leakage.

FIG. 11A is a line graph comparing treatment time in minutes on the x-axis with logarithmic reduction of detected Pichia pastoris on the y-axis. Discharge parameters are noted inset at the top of the chart, set to V=25 kV, I=0.05 mA and d=3.5 cm and varying t from 2.5 min to 30 mins. As shown in FIG. 11A, the log reduction against Pichia pastoris increased from 0.349±0.06 for t=7.5 min to 0.50±0.07 for t=30 min.

FIG. 11B is a bar chart comparing logarithmic reduction of detected Pichia pastoris on the y-axis to three treatment cycles on the x-axis. Discharge parameters are noted inset at the top of the chart, set to V=25 kV, I=0.05 mA and d=3.5 cm. When the sample was treated with n=2 cycles (s=20), the log reduction was increased to 1.041±0.103.

FIG. 11C is a bar chart comparing logarithmic reduction of detected Pichia pastoris on the y-axis to two electrode environments (e.g., open or enclosed) on the x-axis. When the sample was treated in a closed environment (e.g., the right bar) log reduction was increased to 0.96±0.10 for n=1 cycle as shown in FIG. 11C. Ozone decays at a slower rate in closed environment (reaching up to 8.8 ppm after t=20 mins), leading to more oxidative damage to cell membranes as compared to open environment.

Spores were generally harder to disinfect using CD compared to vegetative forms requiring very high discharge voltages (V>50 kV) and long exposure times (t>60 min), see at least Ye, S., et al., tiled Disinfection Of Airborne Spores Of Penicillium expansum In Cold Storage Using Continuous Direct Current Corona Discharge. Biosystems engineering 2012, 113 (2), 112-119, which is incorporated herein by reference in its entirety.

100 μL of E. coli, P. pastoris, and Geobacillus cells were spread on a nutrient agar plate and exposed to CD. FIG. 11D is a bar chart comparing logarithmic reduction of detected microbial cells on the y-axis to three microbial species (e.g., E. coli, P. pastoris, and Geobacillus) on the x-axis. Discharge parameters are noted inset at the top of the chart, discharge parameters being I=0.05 mA, d=3.5 cm, t=7.5 min, n=2, s=20 s, and V=25 kV. A log reduction of detected Geobacillus of 2.52 was observed using the inset discharge parameters. Increased log reduction can be achieved if yeast and spores are treated by wire electrode in closed environment.

Disinfection Mechanism Through DNA and Protein Damage.

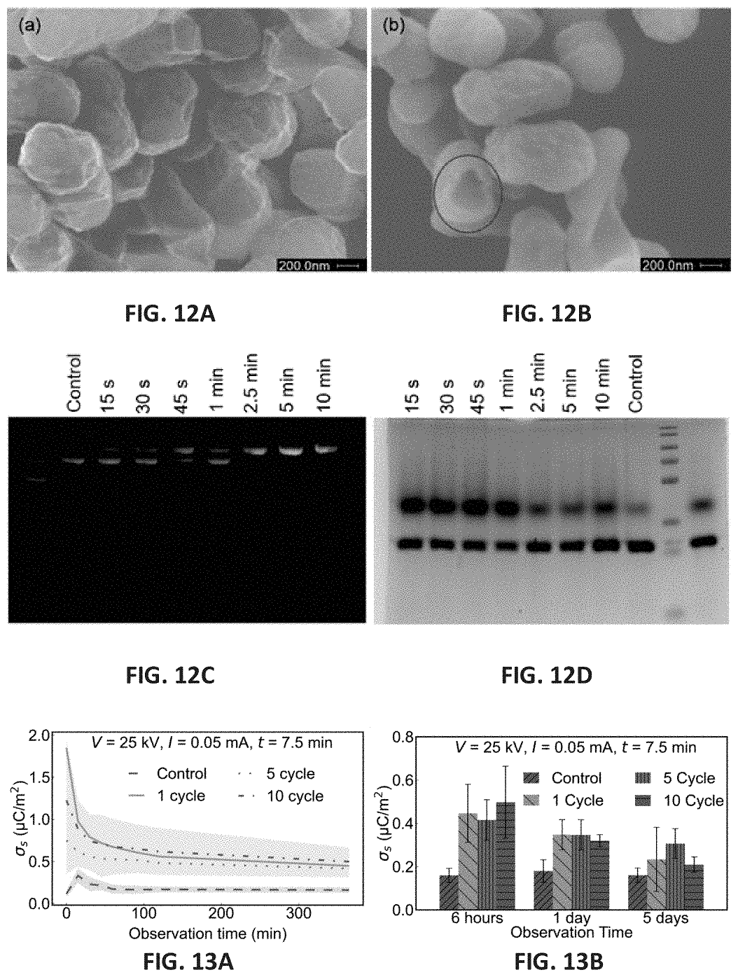

The mechanism of CD bacteria inactivation mechanism was determined through SEM images of global E. coli morphology including CD treatment. FIG. 12A is an SEM image of untreated E. coli with a scale bar representing 200 nm inset at the bottom right. The image of FIG. 12A shows homogenous populations in control condition

FIG. 12B is an SEM image of CD treated E. coli with a scale bar representing 200 nm inset at the bottom right. Cell destruction and pore formation in the cell wall (circled) were observed. The results suggest a non-alteration of the morphology after CD treatment indicating that the inactivation may not be solely due to physical damage caused by ion bombardment or electric field exposure.

It has been previously shown that ROS species induce damage to biomolecules, including DNA and proteins, see at least Timoshkin, I. V. et al., titled Bactericidal Effect of Corona Discharges in Atmospheric Air. IEEE Transactions on Plasma Science 2012, 40 (10), 2322-2333, which is incorporated herein by reference in its entirety. DNA is the genetic material of a cell and damage to the DNA can result in changes in the encoded proteins. These changes may lead to malfunctions or complete inactivation of the encoded proteins as a consequence of molecular alterations.

20 μL of plasmids were exposed to CD treatment (with discharge parameters of V=25 kV, d=3.5 cm and I=0.05 mA), with treatment times ranging from t=15 s tot=10 min and analyzed through agarose gel electrophoresis to determine DNA and protein damage after the exposure. FIG. 12C is a negative image of an agarose electrophoresis gel including nine lanes, each lane corresponding to one sample. From left to right, the samples were a DNA ladder (purchased from Thermo Fisher Scientific), a control sample not exposed to CD treatment, and samples exposed to 15 s, 30 s, 45 s, 1 min, 2.5 min, 5 min, and 10 min, respectively. For all samples but the ladder, two bands are shown: the upper band corresponding to linearized (e.g., broken, non-circular) plasmid DNA, and the lower band corresponding to intact (e.g., circular) plasmid DNA. FIG. 12C shows DNA breakdown initiates at t=2.5 min and the degradation increases as the CD treatment time increases. After t=10 min of CD treatment, all plasmids were in linear form (e.g., non-circular). FIG. 12C depicts double-strand DNA breaking by CD treatment.

A stable fluorescent protein was used in a protein damage experiment in which Superfolder green fluorescent protein (sfGFP) is exposed to CD treatment. sfGFP shows increased thermal stability and genetic fusion tolerance to poorly folding proteins than wild-type (wt)-GFP while remaining fluorescent, see at least Pédelacq, J. D., et al, titled Engineering and Characterization of a Superfolder Green Fluorescent Protein. Nat. Biotechnol. 2006, 24 (1), 79-88 which is incorporated herein by reference in its entirety.

20 μL of sfGFP was exposed to CD (with V=25 kV, d=3.5 cm and I=0.05 mA), then diluted using PBS (1:5 ratio) and samples were disposed in an SDS-PAGE gel and run according to the methods described above. FIG. 12D is an image of an SDS-PAGE electrophoresis gel including ten lanes, each lane corresponding to one sample. From left to right, the samples exposed to 15 s, 30 s, 45 s, 1 min, 2.5 min, 5 min, 10 min, and 20 min, respectively, a control sample not exposed to CD treatment, a protein molecular weight ladder (purchased from Thermo Fisher Scientific), and samples.

For all samples but the ladder, two bands are shown: the upper band corresponding to folded (e.g., stable) sfGFP, and the lower band corresponding to unfolded (e.g., destabilized) sfGFP. Each sample was additionally measured for fluorescence activity as described above. Fluorescence is reduced at t=2.5 min. As CD treatment duration was increased up to 10 min, the proportion of active sfGFP (e.g., the ratio of the upper band to the lower band of FIG. 12D) decreased, resulting in increased inactivation showing that CD caused protein damage (e.g., denaturing).

The interaction of ROS with proteins causes modification of proteins. For example, arginine, lysine, proline and threonine are carbonylated, and histidine modified to oxo-histidine, see at least Ezraty, B., et al., titled Oxidative Stress, Protein Damage and Repair in Bacteria. Nat. Rev. Microbiol. 2017, 15 (7), 385-396, which is incorporated herein by reference in its entirety. Excessive ROS production causes site-specific amino acid modification, fragmentation of the peptide chain, aggregation of cross-linked reaction products, altered electric charge and increased susceptibility of proteins to proteolysis occur, see at least Sharma, P., et al., titled Reactive Oxygen Species, Oxidative Damage, and Antioxidative Defense Mechanism in Plants under Stressful Conditions. Journal of Botany 2012, 2012, 217037, which is incorporated herein by reference in its entirety. FIGS. 12C and 12D show DNA and protein damage, respectively, causing organism inactivation induced by CD treatment.

Recharge Effect on N95 Masks.

High ion density of CD treatment improves the static charge on wearable mask surfaces. These charges improve the filtration efficiency of the mask. N95 (model 8210 manufactured by 3M) masks were exposed to CD treatments including cycle counts of n=1, 5, 10 cycles to show the effect of CD treatment on charge accumulation and decay pattern.

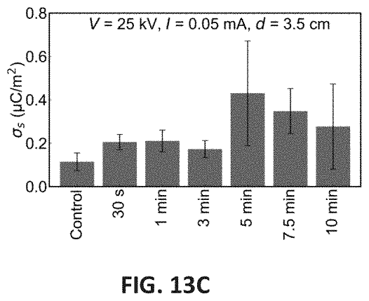

FIG. 13A is a line chart comparing surface charge density (σₛ, μC/m²) on the y-axis to observation time (min) on the x-axis for four cycle counts (e.g., n=0 (control), 1, 5, and 10 cycles). Discharge parameters are noted inset at the top of the chart, discharge parameters being I=0.05 mA, t=7.5 min, s=20 min, and V=25 kV. As shown in FIG. 13A, the maximum σₛ is at t=0 with values as high as σₛ=1.84±0.75 μC/m² after n=1 cycle compared to as =0.11±0.04 μC/m² for untreated control (n=0). The σₛ decreased with increasing observation time for all four cycle counts. The charge density decayed very rapidly during the first 150 min of observation right after treatment due to the release and neutralization of extra free surface charges, after which the “injected” charges were retained. This is similar to previous observation on bulk polymer films, see at least Zhong, Y., et al, titled Electrification Mechanism of Corona Charged Organic Electrets. Journal of Physics D: Applied Physics 2019, 52, 445303 which is incorporated herein by reference in its entirety.

FIG. 13B is a bar chart comparing surface charge density (σₛ, μC/m²) on the y-axis to observation time (days) on the x-axis for four cycle counts (e.g., n=0 (control), 1, 5, and 10 cycles). Discharge parameters are noted inset at the top of the chart, discharge parameters being I=0.05 mA, t=7.5 min, s=20 min, and V=25 kV. Increasing the number of treatment cycles from n=1 to 5 and 10 cycles does not have an effect on σₛ within 1 day of observation (second bar group from left). After 9 days of observation (bar group on right), samples treated with n=5 and 10 cycles exhibited higher charge retention with σₛ=0.185±0.06 and 0.164±0.02 μC/m² respectively while the charges on control and samples treated with n=1 cycle decayed to undetectable levels.

N95 respirators were exposed to CD treatments ranging from t=30 s to 10 min. FIG. 13C is a bar chart comparing surface charge density (σₛ, μC/m²) on the y-axis to treatment time on the x-axis for seven treatment times (e.g., t=0 (control), 30 s, 1 min, 3 min, 5 min, 7.5 min, and 10 min). Discharge parameters are noted inset at the top of the chart, discharge parameters being I=0.05 mA, t=7.5 min, s=20 min, and V=25 kV.

After just 30 s of treatment, the surface charge density (σₛ) reached 0.20±0.03 μC/m² which was well above that of the control sample (σₛ=0.11±0.04 μC/m²). This indicates that the electrostatic charges recovered by CD in N95 respirators can be retained at a functional level for more than 5 days or even longer.

N95 respirators treated with conventional disinfection methods, such as heating, steaming, or pressurization, realize reduced surface charge densities between 52% from 92% of the initial value after exposure to dry heat (70° C.) and high pressures, respectively, see at least Yim, W., et al, titled Kn95 and N95 Respirators Retain Filtration Efficiency Despite a Loss of Dipole Charge During Decontamination, ACS applied materials & interfaces 2020, 12, 54473-54480 which is incorporated herein by reference in its entirety.

As CD treatment recovers N95 masks after 30 s (see FIGS. 13A and 13B), increasing the number of CD treatment cycles and increasing disinfection without decreasing surface charge density. In addition to N95 respirators, CD treatment increase the surface charge density of common face coverings made from natural and/or synthetic fibers and improve their filtration efficiency.

Filtration Efficiency Evaluation.

A standardized filtration efficiency test (e.g., a 1 min loading test derived from NIOSH Procedure No. TEB-APR-STP-0059 using TSI 8130, conducted by Nelson Labs) was performed on N95 respirators and subjected to 15 cycles of CD treatment using the following discharge parameters: V=25 kV, t=7.5 min, s=20 min, in a closed environment, using a 5 cm long tungsten wire electrode. The resulting filtration efficiency of multiple samples was confirmed to be 94.46±0.372%, see Table 1.

TABLE 1

| | Corrected^{a} Airflow | Particle Penetration | Filtration |
|---|---|---|---|
| Test Article | Resistance (mm H₂O) | (%) | Efficiency |
| JO531 | 6.4 | 5.58 | 94.42 |
| JO532 | 6.9 | 5.14 | 94.86 |
| JO533 | 6.7 | 5.88 | 94.12 |

^{a}The final airflow resistance value for each test article was determined by subtracting out the background resistance from the system.

This result indicates that the filtration efficiency of N95 respirators were similar to the untreated control respirators without deteriorated filtration efficiency following 15 cycles of CD treatment. The number of reuses for N95 respirators can be extended to at least 10 by CD treatment, more than following other disinfection solutions (e.g., solvent dip or spray).

High voltage and low current is utilized (e.g., V=25 kV, I≤0.05 mA) resulting in low CD treatment power consumption (e.g., less than 1.25 W). Portable CD treatment devices can be designed and manufactured which utilize commercial hand-held high voltage transformers and power supplies. Ozone and UV radiation generated by CD treatment are lower than current ozone generators or UV lights used in alternative disinfection methods, indicating that CD treatment is an energy efficient and safe solution of disinfecting and charge injecting N95 respirators for reuse.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating an article, the method comprising:
generating a corona discharge from an electrode;
providing an article within the corona discharge; and
exposing the article to the corona discharge to disinfect and/or electrically charge the article.

2. The method of claim 1, wherein generating comprises applying a voltage to the electrode of less than 100 kV.

3. The method of claim 2, wherein generating comprises applying a voltage selected from the group consisting of about 6 kV to about 40 kV, about 6 kV to about 10 kV, about 10 kV to about 20 kV, or about 20 kV to about 40 kV.

4. The method of claim 1, wherein exposing occurs in a cycle, each cycle including a treatment time and a storage time, wherein the article is exposed to the corona discharge during the treatment time, and wherein the article is not exposed to the corona discharge during the storage time.

5. The method of claim 4, wherein the treatment time is from about 15 s to about 10 mins.

6. The method of claim 4, wherein the storage time is from about 1 s to 60 min.

7. The method of claim 4, wherein the exposing occurs in 2 cycles to 10 cycles.

8. The method of claim 1, wherein the exposing reduces a detectable biologic marker by a logarithmic power value in a range from 0.5 to 8.

9. The method of claim 1, wherein the article is a mask or a filter.

10. The method of claim 1, wherein the exposing reduces or eliminates the presence of a bacteria, a spore, a fungus, a virus, or combinations thereof.

11. The method of claim 1, wherein the exposing increases a surface charge density of the article by at least 0.001 μC/m².

12. The method of claim 11, wherein the surface charge density of the article is increased for at least 1 days.

13. The method of claim 1, wherein the exposing comprises generating an ion density of about 10⁴ to about 10⁸ ions/cm³.

14. The method of claim 1, wherein the providing comprises arranging the article or portions of the article between about 1 cm to about 50 cm from the electrode.

15. A system for treating an article, the system comprising:
a power supply or a means for connecting to a power supply; and
a discharge electrode electrically coupled to the power supply;
wherein the system is configured to generate a corona discharge from the discharge electrode to disinfect and/or electrically charge the article.

16. The system of claim 15, wherein the discharge electrode comprises a needle, a plurality of needles, a wire, a plurality of wires, or a flat plate.

17. The system of claim 15, wherein the discharge electrode has a tip having a curvature radius of about 0.1 μm to about 1500 μm.

18. The system of claim 15, wherein the system comprises a portable device, wherein the portable device is a hand-held device including one or more nozzles, each nozzle comprising the discharge electrode, and wherein the hand-held device comprising a handle configured for single hand.

19. The system of claim 15, wherein the system comprises an enclosure comprising a housing configured for receiving the discharge electrode and a sample support structure configured to receive the article, wherein the discharge electrode and the article on the sample support structure are arranged within the enclosure housing such that the article is spaced apart from the discharge electrode by an electrode-sample distance.

20. The system of claim 19, wherein the discharge electrode comprises a wire shaped in the form of a ring, and wherein the system comprises an electrode support structure configured to receive and position the wire by the electrode-sample distance from the article.
